(19) United States
(12) Patent Application Publication
(10) Pub. No.: US 2010/0204341 A1
(43) Pub. Date: Aug. 12, 2010

(54) PERSONAL CARE DISSOLVABLE FILMS

(76) Inventors: Wei Hong Yu, Edison, NJ (US); Tatiana V. Drovetskaya, Basking Ridge, NJ (US); Susan L. Jordan, Doylestown, PA (US)

Correspondence Address: UNION CARBIDE CHEMICALS AND;PLASTICS TECHNOLOGY CORPORATION, P.O. Box 1967, Midland, MI, 48641-1967, US

(21) Appl. No.: 12/665,266

(22) PCT Filed: Jun. 26, 2008
(86) PCT No.: PCT/US08/68374

§ 371 Date: Apr. 12, 2010

(30) Foreign Application Priority Data
Jun. 29, 2007 (US) 60947123

(51) Int. Cl. A61K 47/02; A61K 47/32; A61K 47/36; A61K 47/38; A61K 47/24; A61K 47/44; A61K 47/10; A61Q 1/02

(52) U.S. Cl. 514/770; 514/772.6; 514/772.4; 514/777; 514/781; 514/778

(57) ABSTRACT

Personal care dissolvable films comprising a water soluble film forming agent, cosmetically acceptable plasticizer, and a thickener are described, along with methods of using the same.

### FIELD

The present invention relates to personal care products and methods of making and using the same.

### BACKGROUND

Personal care films offer a convenient platform for delivering compositions, such as for skin care, hair care, or cosmetics applications. Such films are designed to quickly dissolve upon contact with water or aqueous compositions provided by the end user. An important goal in the industry is that the performance of these films will rival that of their conventional wet counterparts, while providing the greater portability and convenience of a film.

The success of a cosmetic, including personal care films, depends in great measure on the way it feels to a user. Thus, speed of dissolution and sensory attributes, such as ease of spreading, speed of absorption, texture, skin moistness, heaviness, amount of grease, amount of tack, quickness of drying, overall skin feel, and overall appearance are critical factors. For example, if the product dissolves too slowly, it becomes gummy and lumpy and does not provide uniform film. In the past, personal care films have been developed that dissolve quickly, but have not attained tactile properties that would lead to a successful product.

Thus, what is needed are personal care films with even more desirable consumer properties.

### SUMMARY

In one embodiment, the present invention provides personal care dissolvable films comprising a water soluble film forming agent, a cosmetically acceptable plasticizer, and a thickener.

### DETAILED DESCRIPTION

In one embodiment, the present invention provides a personal care dissolvable film, comprising a water soluble film forming agent, a cosmetically acceptable plasticizer, and a thickener.

“Personal care” refers to compositions that are to be topically applied to a person. Examples of personal care compositions include skin care and cosmetic products (e.g., facial cream, moisturizers, lotions, sunscreens, foundation, mascara, eye-liner, lipstick, and the like), nail care products (such as polish and conditioners), and hair care products (including styling gels and hairsprays). A “personal care dissolvable film” is a readily dissolvable film containing a personal care composition. In one embodiment, such a film dissolves in less than a minute when wet with water or aqueous compositions and rubbed between fingers for example.

“Cosmetically acceptable” refers to ingredients typically used in personal care compositions, and is intended to underscore that materials that are toxic, irritating, or unpleasant smelling when present in the amounts typically found in personal care compositions are not contemplated as part of the present invention.

The term “water-soluble film forming agent” includes polymers with a solubility in water, measured at 25° C., of at least 0.1 gram/liter (g/L). This solubility is preferably at least 1 g/L. The polymers may be synthetic, natural, or modified, and include wheat or soybean proteins, keratin, for example keratin hydrolysates and sulfonic keratins, casein, albumin, collagen, glutelin, glucagon, gluten, zein, gelatins and derivatives thereof, polymers derived from chitin or from chitosan, such as anionic, cationic, amphoteric or nonionic chitin or chitosan polymers, polysaccharide polymers such as cellulose-based polymers, for instance hydroxyethylcellulose, hydroxypropylcellulose, hydroxypropylmethylcellulose, methylcellulose, ethylhydroxyethylcellulose, carboxymethylcellulose and quaternized cellulose derivatives, starches and derivatives thereof, acrylic polymers or copolymers such as polyacrylates, polymethacrylates and copolymers thereof, vinyl polymers such as polyvinylpyrrolidones, copolymers of methyl vinyl ether and of maleic anhydride, the copolymer of vinyl acetate and of crotonic acid, copolymers of vinylpyrrolidone and of vinyl acetate, copolymers of vinylpyrrolidone and of caprolactam, polyvinyl alcohols, polymers of natural origin, which are optionally modified, such as gum arabic, guar gum, xanthan derivatives or karaya gum, alginates, carrageenans, ulvanes and other algal colloids, glycoaminoglycans, hyaluronic acid and its derivatives, shellac, sandarac gum, dammar resins, elemi gums and copal resins, deoxyribonucleic acid, mucopolysaccharides such as hyaluronic acid, chondroitin sulphate, caprolactams, pullulan, pectin, mannan and galactomannans, and glucomannans, and mixtures and/or derivatives thereof.

In one embodiment, the water soluble film forming agent is a cellulose ether based polymer, polyethylene oxide, or mixtures thereof. In one embodiment, the water soluble film forming agent is at least one of methylcellulose, hydroxypropyl methylcellulose, hydroxyethyl cellulose, cationic hydroxyethyl cellulose, hydrophobically modified hydroxyethyl cellulose, or cationic hydrophobically modified hydroxyethyl cellulose. In one embodiment, the water soluble film forming agent is hydroxypropyl methylcellulose.

The water soluble film forming agent is present in an amount from about 1% to about 50% by weight of the film forming composition. The “film forming composition” refers to the composition that is cast, extruded, or otherwise processed, to become the personal care dissolvable film. Typically, water or other suitable liquid will comprise the majority of the film forming composition. Preferably, the water soluble film forming agent is present in an amount from about 5% to about 20% by weight of the film forming composition. In one embodiment, the water soluble film forming agent is present in an amount greater than about 7%, preferably greater than about 9%, more preferably greater than about 11%, and preferably about 14%.

In one embodiment, the film forming composition contains less than 5 wt. % surfactant. In one embodiment, the film forming composition contains less than 3 wt. % surfactant, and preferably less than 1 wt. %.

In one embodiment, the film has a dissolution rate of at least about 3 to about 10 seconds, using the following protocol. A 2-3 mil, 2 cm×1.5 cm, film is weighed. The weight should be about 0.0175 g to about 0.0225 g depending on thickness. It is understood that the dissolution rate will be affected by thickness, all other factors being equal. The film is placed in the palm of a tester's hand and about 2.5 mL of water are added. With a finger, the wetted film is rubbed in a circular motion, two circles representing a second, until the film is dissolved.

In one embodiment, the plasticizer is at least one of a polyalcohol, including glycerin, glycerol, propylene glycol, sorbitol, dipropylene glycol, butylene glycol, pentylene glycol, and polyethylene glycols (including PEG 400), mono- and/or disaccharides, or polyethylene oxide.

In one embodiment, the plasticizer is present in an amount from about 0.01% to about 40.0% by weight of the film forming composition. Preferably, the plasticizer is present in an amount from about 2% to about 15.0% by weight of the film forming composition. In one embodiment, the plasticizer is present in an amount greater than about 2%, preferably greater than about 4%, more preferably greater than about 5%, and preferably about 6%.

In one embodiment, the plasticizer is a polyalcohol and the film also contains skin actives selected from the group consisting of chitosan/pyrrolidone carboxylic acid mixture and polyquaternium-24 hyaluronate. In this embodiment, the plasticizer may be present in an amount from about 5.0% to about 20.0% by weight of the film forming composition, and preferably from about 7.0% to about 17.0%.

In one embodiment, the thickener is at least one of carboxyvinyl polymers, such as the products sold under the names CARBOPOL and PEMULEN (INCI name: Acrylates/C₁₀₋₃₀ alkyl acrylate crosspolymer; available from Noveon), polyacrylates & polymethacrylates, such as the products sold under the names LUBRAJEL and NORGEL (from Guardian) or HISPAGEL (from Hispano Chimica), polyacrylamides, and sodium polyacrylate/dimethicone/cyclopentasiloxane/tri-deceth-6/PEG-PPG-18/18 dimethicone, polyacrylamides, for example, polyacrylamide/C13-C14 isoparaffin/laureth-72-acrylamido-2-methylpropanesulfonic acid polymers and copolymers, poly(2-acrylamido-2-methylpropane-sulfonic acid) sold by Clariant (INCI name: ammonium polyacryldimethyltauramide), emulsified crosslinked anionic copolymers of acrylamide and AMPS, such as those sold under the name SEPIGEL 305 (INCI name: Polyacrylamide/C13-14 Isoparaffin/Laureth-7; from Seppic) and under the name SIMULGEL 600 (INCI name: Acrylamide/Sodium acryloyldimethyltaurate copolymer/Isohexadecane/Polysorbate 80; from Seppic), polysaccharide biopolymers, for instance xanthan gum, guar gum, carob gum, acacia gum, scleroglucans, chitin and chitosan derivatives, carrageenans, gellans, alginates, starches, associative polymers, associative polyurethanes, copolymers comprising at least two hydrocarbon-based lipophilic chains comprising from 6 to 30 carbon atoms, separated with a hydrophilic sequence, such as the polyurethanes sold under the names SERAD FX1010, SERAD FX1100 and SERAD FX1035 (from Hüls America), RHEOLATE 255, RHEOLATE 278 and RHEOLATE 244 (INCI name: Polyether-urea-polyurethane; from Rheox), DW 1206F, DW 1206J, DW 1206B, DW 1206G, and ACRYSOL RM 2020 (from Röhm & Haas), water-soluble vinyl polymer, and celluloses such as microcrystalline cellulose, carboxymethylcellulose, hydroxymethylcellulose and hydroxypropylcellulose, provided that the cellulose used as a thickener is additional to the above-mentioned water soluble film forming agent (when the latter is also a cellulose ether based polymer). Generally, a relatively higher molecular weight grade of cellulose, i.e., greater than about 80,000 (Mn), will find use primarily as a thickener.

In a preferred embodiment, the thickener is a mixture of polyacrylamide, C13-14 isoparaffin, and LAURETH-7. In another preferred embodiment, the thickener is a mixture of sodium polyacrylate/dimethicone/cyclopentasiloxane/tri-deceth-6/PEG-PPG-18/18 dimethicone. In yet another preferred embodiment, the thickener is an acrylates/C10-30 alkyl acrylate crosspolymer.

In one embodiment, the thickener is a mixture of at least two of a mixture of polyacrylamide, C₁₃₋₁₄ isoparaffin, and LAURETH-7, a mixture of sodium polyacrylate/dimethicone/cyclopentasiloxane/tri-deceth-6/PEG-PPG-18/18 dimethicone, and an acrylates/C10-30 alkyl acrylate crosspolymer.

In one embodiment, the thickener is present in an amount from about 0.01% to about 10% by weight of the composition. In one embodiment, the thickener is present in an amount from about 0.1% to about 4% by weight of the composition. In one embodiment, the thickener is present in an amount greater than about 0.4%, preferably greater than about 0.6%, and more preferably greater than about 0.9%.

When the thickener is a carboxyvinyl polymer, the thickener may be present in an amount from about 0.1% to about 0.2% by weight of the composition.

When the thickener is a polyacrylate, the thickener is present in an amount from about 1% to about 2% by weight of the composition.

In one embodiment, the present invention provides personal care dissolvable films comprising hydroxypropyl methylcellulose as a water soluble film forming agent. In one embodiment, this film further comprises propylene glycol as a plasticizer.

In one embodiment, the present invention further includes optional ingredients. The amount of optional ingredients effective for achieving the desired property provided by said ingredients can be readily determined by one skilled in the art.

In one embodiment, the present invention includes an optional detackifier. Detackifiers contemplated in the present invention include, for example, silicone elastomers, calcium aluminum borosilicate, silica, alumina, boron nitride, talc (which may be coated or uncoated), composite fillers, powder or aqueous dispersions of polytetrafluoroethylene (PTFE), wax microdispersions, polyvinylpyrrolidone/1-tricontene copolymers, silicone waxes and resins, organopolysiloxane particles, microspheres of expanded terpolymer of vinylidene chloride, acrylonitrile and methacrylate, cellulose microbeads, fibres, hollow hemispherical silicone particles such as NLK-500 and NLK-503 (from Takemoto Oil and Fat). Other suitable detackifiers include polyamide particles, preferably NYLON 12 powder, such as ORGASOL (from Atochem), polyethylene powders and beads such as ACUMIST B-6 or B-12 (from Allied) and MICROTHENE (from Equistar), microspheres based on acrylic or methacrylic copolymers, such as those made of ethylene glycol dimethacrylate/lauryl methacrylate copolymer, such as POLYTRAP (from Dow Corning), methyl methacrylate/ethylene glycol dimethacrylate copolymer, such as MICROSPHERES M-305 or M-100 (from Matsumoto), polymethyl methacrylate microspheres such as COVABEAD (from Wackherr), ethylene-acrylate copolymer powders, such as FLOBEADS (from Sumitomo Seika Chemicals), expanded powders such as hollow microspheres, such as EXPANCEL (from Kemanord Plast) or MICROPEARL F 80 ED (from Matsumoto), powders of natural organic materials such as corn, wheat, or rice starches, crosslinked or otherwise, including DRY-FLO starch which is crosslinked with octenyl succinate anhydride (from National Starch), microbeads of silicone resin such as TOSPEARL (from GE Bayer Silicones), and mixtures thereof.

In one embodiment, the detackifying agent is at least one of dimethicone crosspolymer, calcium aluminum borosilicate spheres, or silicone resin particles including siloxane bonds and silicone groups bonded to methyl groups.

In some embodiments, the detackifying agent is a solid before addition to the film forming composition. In some embodiments, the detackifying agent is a liquid before addition to the film forming composition. Such embodiments can optionally include a thickener to achieve the desired consistency.

In one embodiment, the detackifying agent is present in an amount from about 0.05% to about 25% by weight of the film forming composition. In one embodiment, the detackifying agent is present in an amount from about 0.1% to about 5% by weight of the film forming composition.

In one embodiment, the personal care compositions of the present invention further comprise an active ingredient selected from skin care actives, nail care actives, or hair care actives. Actives include emollients, moisturizers, conditioners, anti-aging agents, sunscreens, skin colorants, drug substances (such as anti-inflammatory agents, antibiotics, topical anesthetics, antimycotics, keratolytics, and the like), skin protectants, humectants, and ultraviolet radiation absorbers.

Examples of sunscreens include paraminobenzoic acid, avobenzone, cinoxate, dioxybenzone, homosalate, menthyl anthranilate, octocrylene, octyl methoxycinnamate, octyl salicylate, oxybenzone, padimate O, phenylbenzimidazole sulfonic acid, sulisobenzone, trolamine salicylate, titanium dioxide and zinc oxide, diethanolamine methoxycinnamate, digalloy trioleate, ethyl dihydroxypropyl PABA, glyceryl aminobenzoate, lawsone with dihydroxy acetone, and red petrolatum.

In one embodiment, the active is a moisturizer or anti-aging additive. Preferably, the active is selected from chitosan/pyrrolidone carboxylic acid mixtures and/or polyquatemium-24 hyaluronates. Examples of these skin actives include KYTAMER PC chitosan/pyrrolidone carboxylic acid mixture and/or BIOCARE HA-24 polyquaternium-24 hyaluronate, each available from The Dow Chemical Company. The moisturizers also include 2-pyrrolidone-5-carboxylic acid and its salts and esters, alkyl glucose alkoxylates or their esters, fatty alcohols, fatty esters, glycols and, in particular, methyl glucose ethoxylates or propoxylates and their stearate esters, isopropyl myristate, lanolin or cetyl alcohols, aloe, silicones, propylene glycol, glycerol and sorbitol.

Conditioners include stearalkonium chloride, dicetyldimonium chloride, lauryl methyl gluceth-10 hydroxypropyldimonium chloride, and conditioning polymers such as polyquaternium-10, polyquatemium-24 and chitosan and derivatives thereof.

Emollients include oils or other fatty substances. The term “oil” means a fatty substance that is liquid at room temperature. Examples of oils include hydrocarbon-based oils of animal origin, such as squalene, hydrocarbon-based oils of plant origin, such as liquid triglycerides of fatty acids comprising from 4 to 10 carbon atoms, for instance heptanoic or octanoic acid triglycerides, or alternatively, oils of plant origin, for example sunflower oil, corn oil, soybean oil, marrow oil, grapeseed oil, sesame seed oil, hazelnut oil, apricot oil, macadamia oil, arara oil, coriander oil, castor oil, avocado oil, jojoba oil, shea butter oil, or caprylic/capric acid triglycerides, MIGLYOL 810, 812 and 818 (from Dynamit Nobel), synthetic esters and ethers, especially of fatty acids, for instance the oils of formulae R¹COOR² and R¹OR² in which R¹ represents a fatty acid residue comprising from 8 to 29 carbon atoms and R² represents a branched or unbranched hydrocarbon-based chain comprising from 3 to 30 carbon atoms, for instance purcellin oil, isononyl isononanoate, isopropyl myristate, 2-ethylhexyl palmitate, 2-octyldodecyl stearate, 2-octyldodecyl erucate or isostearyl isostearate, hydroxylated esters, for instance isostearyl lactate, octyl hydroxystearate, octyldodecyl hydroxystearate, diisostearyl malate, triisocetyl citrate and fatty alcohol heptanoates, octanoates and decanoates, polyol esters, for instance propylene glycol dioctanoate, neopentyl glycol diheptanoate and diethylene glycol diisononanoate, pentaerythritol esters, for instance pentaerythrityl tetraisostearate, lipophilic derivatives of amino acids, such as isopropyl lauroyl sarcosinate, such as is sold under the name ELDEW SL 205 (from Ajinomoto), linear or branched hydrocarbons of mineral or synthetic origin, such as mineral oils (mixtures of petroleum-derived hydrocarbon-based oils), volatile or non-volatile liquid paraffins, and derivatives thereof, petroleum jelly, polydecenes, isohexadecane, isododecane, hydrogenated isoparaffin (or polyisobutene), silicone oils, for instance volatile or non-volatile polymethylsiloxanes (PDMS) comprising a linear or cyclic silicone chain, which are liquid or pasty at room temperature, especially cyclopolydimethylsiloxanes (cyclomethicones) such as cyclopentasiloxane and cyclohexadimethylsiloxane, polydimethylsiloxanes comprising alkyl, alkoxy or phenyl groups, which are pendent or at the end of a silicone chain, these groups comprising from 2 to 24 carbon atoms, phenyl silicones, for instance phenyl trimethicones, phenyl dimethicones, phenyltrimethylsiloxydiphenylsiloxanes, diphenyl dimethicones, diphenylmethyldiphenyltrisiloxanes 2-phenylethyltrimethyl siloxysilicates and polymethylphenylsiloxanes, fluoro oils such as partially hydrocarbon-based and/or partially silicone-based fluoro oils, ethers such as dicaprylyl ether (CTFA name: dicaprylyl ether), and C₁₂-C₁₅ fatty alcohol benzoates (FINSOLV TN from Finetex), mixtures thereof.

In one embodiment, oils include mineral oil, lanolin oil, coconut oil and derivatives thereof, cocoa butter, olive oil, almond oil, macadamia nut oil, aloe extracts such as aloe vera lipoquinone, jojoba oils, safflower oil, corn oil, liquid lanolin, cottonseed oil, peanut oil, hydrogenated vegetable oil, squalane, castor oil, polybutene, sweet almond oil, avocado oil, calophyllum oil, ricin oil, vitamin E acetate, olive oil, silicone oils such as dimethylopolysiloxane and cyclomethicone, linolenic alcohol, oleyl alcohol, and the oil of cereal germs.

Other suitable emollients include dicaprylyl ether, C₁₂₋₁₅ alkyl benzoate, DC 200 FLUID silicone fluids (from Dow Corning Corp.), isopropyl palmitate, octyl palmitate, isopropyl myristate, hexadecyl stearate, butyl stearate, decyl oleate, acetyl glycerides, the octanoates and benzoates of C₁₂₋₁₅ alcohols, the octanoates and decanoates of alcohols and polyalcohols such as those of glycol and glyceryl, ricinoleates esters such as isopropyl adipate, hexyl laurate and octyl dodecanoate, dicaprylyl maleate, phenyltrimethicone, and aloe vera extract. Solid or semi-solid cosmetic emollients include glyceryl dilaurate, hydrogenated lanolin, hydroxylated lanolin, acetylated lanolin, petrolatum, isopropyl lanolate, butyl myristate, cetyl myristate, myristyl myristate, myristyl lactate, cetyl alcohol, isostearyl alcohol and isocetyl lanolate.

In one embodiment, the emollient is PPG-14 butyl ether.

In one embodiment, the emollient is present in an amount from about 0.5% to about 20% by weight of the film forming composition.

Additional optional ingredients include any suitable substance for personal care compositions, for example, colorants, preservatives, pH adjustors, reducing agents, fragrances, foaming agents, tanning agents, depilatory agents, flavors, astringents, antiseptics, deodorants, antiperspirants, insect repellants, bleaches and lighteners, anti-dandruff agents, adhesives, polishes, strengtheners, fillers, barrier materials, and biocides.

Colorants include pigments, which are used especially in make-up, including metal oxide pigments, titanium dioxide, optionally surface-treated, zirconium oxide or cerium oxide, zinc oxide, iron oxide (black, yellow or red), chromium oxide, manganese violet, ultramarine blue, chromium hydrate and ferric blue, carbon black, pigments of barium, strontium, calcium or aluminum (for example D&C or FD&C), cochineal carmine, mica coated with titanium or with bismuth oxychloride, titanium mica with iron oxides, titanium mica with, especially, ferric blue or chromium oxide, titanium mica with an organic pigment, nacreous pigments based on bismuth oxychloride, goniochromatic pigments, for example pigments with a multilayer interference structure, reflective pigments, for example particles with a silver-coated glass substrate, glass substrate coated with nickel/chromium/molybdenum alloy, glass substrate coated with brown iron oxide, particles comprising a stack of at least two polymer layers, for instance MIRROR GLITTER (from 3M).

Dyes include water-soluble dyes such as copper sulfate, iron sulfate, water-soluble sulfopolyesters, rhodamines, natural dyes, for instance carotene and beetroot juice, methylene blue, caramel, the disodium salt of tartrazine and the disodium salt of fuschin, and mixtures thereof. Liposoluble dyes may also optionally be used.

Preservatives include alcohols, aldehydes, methylchloroisothiazolinone and methylisothiazolinone, p-hydroxybenzoates, and in particular methylparaben, propylparaben, glutaraldehyde and ethyl alcohol.

The pH adjustors, include inorganic and organic acids and bases and in particular aqueous ammonia, citric acid, phosphoric acid, acetic acid, and sodium hydroxide.

Reducing agents include ammonium thioglycolate, hydroquinone and sodium thioglycolate.

Fragrances include any component which provides a pleasant scent. Fragrances are generally aldehydes or ketones, and often oils obtained by extraction of natural substances or synthetically produced. Often, fragrances are accompanied by auxiliary materials, such as fixatives, extenders, stabilizers and solvents.

Biocides include antimicrobials, bactericides, fungicides, algaecides, mildicides, disinfectants, antiseptics, and insecticides.

The present invention further includes methods of using the above-described personal care dissolvable films, comprising wetting the film with water or an aqueous composition and applying it to a person. In one embodiment, the film is wetted and rubbed between fingers to produce a personal care composition.

In yet another embodiment, the present invention provides an eye care composition dissolvable film, comprising a water soluble film forming agent, a cosmetically acceptable plasticizer, and a thickener.

### EXAMPLES

The following examples are for illustrative purposes only and are not intended to limit the scope of the present invention. All percentages are by weight unless otherwise specified.

### Example 1

Exemplary personal care dissolvable films contain the components recited in TABLE 1. “%” indicates percent by weight of film-forming composition.

TABLE 1

| | | Batch 1 | Batch 2 |
|---|---|---|---|
| | Component | % | % |
| A | Propylene Glycol | 2.0 | 8.0 |
| | METHOCEL E3 Hydroxylpropyl methylcellulose | 14.0 | 14.0 |
| | (available from The Dow Chemical Company) | | |
| | KYTAMER 5% solution chitosan/pyrrolidone | 7.6 | 7.6 |
| | carboxylic acid mixture | | |
| | (available from The Dow Chemical Company) | | |
| | POLYOX N750 polyethylene oxide | 0.02 | 0.02 |
| | (available from The Dow Chemical Company) | | |
| | Water | 73.26 | 62.26 |
| B | UCON AP PPG-14 Butyl Ether | 1.0 | — |
| | (available from The Dow Chemical Company) | | |
| C | BIOCARE HA-24 polyquaternium-24 hyaluronate | 0.08 | 0.08 |
| | (available from The Dow Chemical Company) | | |
| | NIPAGUARD propylene glycol, diazolidinyl | 0.04 | 0.04 |
| | urea, methyl paraben, propyl paraben | | |
| | preservative (available from Clariant) | | |
| D | DDS 524-MB dimethicone crosspolymer/ | 2.0 | 2.0 |
| | Laureth-4/Laureth-23/water | | |
| | (available from The Dow Chemical Company) | | |
| | UCON AP PPG-14 Butyl Ether | — | 4.0 |
| | (available from The Dow Chemical Company) | | |
| | SEPIGEL 305 polyacrylamide/C13-14 | — | 2.0 |
| | Isoparaffin/Laureth-7 | | |
| | (available from Seppic, Inc.) | | |

Part A ingredients are combined and mixed at a temperature of about 90° C. until the polymer completely hydrates and dissolves. Then the Part A is cooled to room temperature. Part B ingredients are added to Part A one by one and mixed until the dispersion is uniform. The rest of ingredients are added to the batch in order and mixed until uniform. The resulting composition is cast via conventional methods.

### Example 2

Exemplary personal care dissolvable films contain the components recited in TABLE 2. “%” indicates percent by weight of film-forming composition.

TABLE 2

| | Component | Batch 3 | Batch 4 | Batch 5 |
|---|---|---|---|---|
| A | Propylene Glycol | 6.00 | 6.00 | 6.00 |
| | METHOCEL E3 Hydroxylpropyl | 14.00 | 14.00 | 14.00 |
| | methylcellulose (available from The | | | |
| | Dow Chemical Company) | | | |
| | water | 66.71 | 67.11 | 68.11 |
| B | SEPIGEL 305 polyacrylamide/C13-14 | 1.40 | 1.00 | 1.00 |
| | Isoparaffin/Laureth-7 (available from | | | |
| | Seppic, Inc.) | | | |
| C | 69% Titanium Dioxide/isononyl | 8.00 | 8.00 | 8.00 |
| | isononanoate | | | |
| | 68.6% red Iron Oxide/isononyl | 0.19 | 0.19 | 0.19 |
| | isononanoate | | | |
| | 53.9 Yellow Iron Oxide/isononyl | 1.50 | 1.50 | 1.50 |
| | isononanoate | | | |
| | 68.5% Black Iron Oxide/isononyl | 0.16 | 0.16 | 0.16 |
| | isononanoate | | | |
| D | LUXSIL calcium aluminum borosilicate | 2.00 | | |
| | spheres (available from Potters | | | |
| | Industries, Inc.) | | | |
| | DC 9509 POWDER SUSPENSION | | 2.00 | 1.00 |
| | Dimethicone/Vinyl Dimethicone | | | |
| | Crosspolymer (and) C12-14 Pareth-12 | | | |
| | (available from Dow Corning) | | | |
| | NIPAGUARD propylene glycol, | 0.04 | 0.04 | 0.04 |
| | diazolidinyl urea, methyl paraben, | | | |
| | propyl paraben preservative (available | | | |
| | from Clariant) | | | |

Part A ingredients are combined and mixed at a temperature of about 90° C. until the polymer completely hydrates and dissolves. Then the Part A is cooled to room temperature. Part B and Part C ingredients are added to Part A one by one and mixed until the dispersion is uniform. The rest of ingredients are added to the batch in order and mixed until uniform. The resulting composition is cast via conventional methods.

### Example 3

A film forming composition was made substantially according to the protocol described in Example 2, Batch 5, and a panelist study was conducted. NEUTROGENA VISIBLY EVEN foundation was used as a comparison for sensory properties, as no known commercial dissolvable film used as foundation exists.

Ten panelists applied 0.05 g of each sample to a designated area on their right or left forearms. At initial application, each sample was evaluated for ease of spread, evenness of color, slip, tackiness, coverage, speed of absorption, quickness of drying, overall skin feel, and overall appearance. The evaluation scale was 0-10, with 10 being outstanding, and the results are displayed in TABLE 3A.

TABLE 3A

| | | NEUTROGENA VISIBLY |
|---|---|---|
| | Batch 5 | EVEN foundation |
| Ease of Spread | 5.90 | 6.40 |
| Evenness of Color | 5.70 | 6.60 |
| Slip | 5.80 | 4.70 |
| Tackiness | 5.80 | 4.90 |
| Coverage | 5.20 | 6.60 |
| Speed of Adsorption | 6.90 | 5.70 |
| Drying Quickly | 7.20 | 5.30 |
| Overall Skin Feel | 6.50 | 5.70 |
| Overall Appearance | 6.50 | 6.20 |

As seen in the table, Batch 5 out-performed a commercial “wet” product for slip, speed of adsorption, drying quickly, less tacky and overall skin feel. And, the overall appearance of the Batch 5 formula is slightly better or the same as the conventional product. Generally, a dissolvable film product would be expected to have unpleasant tactile properties due to the high quantity of film forming polymer. One of the major drawbacks of previously known film products was the poor sensory properties. Additionally, it is difficult to achieve quick dissolving films for leave-on products because of the low concentration of surfactant in the formula. However, the presently claimed films overcome these problems.

After one hour, each sample was evaluated for coverage, evenness of color, skin softness, tackiness, overall skin feel, and overall appearance. The results are displayed in TABLE 3B.

TABLE 3B

| | | NEUTROGENA VISIBLY |
|---|---|---|
| | Batch 5 | EVEN foundation |
| Coverage | 5.8 | 6.7 |
| Evenness of Color | 6.2 | 6.5 |
| Skin Softness | 6.1 | 6.2 |
| Tackiness | 7.3 | 6.5 |
| Overall Skin Feel | 6.7 | 6.0 |
| Overall Appearance | 5.5 | 5.9 |

As seen in the table, Batch 5 out-performed a commercial wet product for less tacky and overall skin feel. Other attributes are close to the conventional product. When the portability of a film versus a wet product is considered, the results are very significant.

### Example 4

Films made substantially according to the protocol of Example 1 and Example 2 representing Batch 1 and Batch 3 were compared to commercially available OSIS SHOCKFROSTER hair styling strips (modified corn starch, PVP, water, propylene glycol, octylacrylamide/acrylates/butylaminoethyl/methacrylate copolymer, aminomethyl propanol, aluminum starch octenylsuccinate, fragrance, benzyl salicylate, limonene, butylphenyl methylproprional, linalool, and Red 40) for dissolution rates.

Dissolution rates were measured using the Hand Rubbed Dissolution Test that simulates real-life usage conditions. A 2 cm.×1.5 cm piece of dissolvable film is placed in the palm of the operator's left hand 2.5 ml of water are added and the operator rubs the film with the water using two fingers of the right hand in a circular motion (each circle taking approximately one second) until the film is completely dissolved. The dissolution times (average of two measurements) are determined.

Using the Hand Rubbed Dissolution Test, Batch 1 dissolved in 3 (±2) seconds; Batch 3 also dissolved in 3 (±2) seconds; OSIS SHOCKFROSTER hair styling strips dissolved in 15 (±2) seconds. Thus, films of the present invention performed significantly better. Any improvement in dissolution time is important, as a relatively faster dissolution time relates to positive consumer experience.

It is understood that the present invention is not limited to the embodiments specifically disclosed and exemplified herein. Various modifications of the invention will be apparent to those skilled in the art. Such changes and modifications may be made without departing from the scope of the appended claims.

Moreover, each recited range includes all combinations and subcombinations of ranges, as well as specific numerals contained therein. Additionally, the disclosures of each patent, patent application, and publication cited or described in this document are hereby incorporated herein by reference, in their entireties.

1. A personal care dissolvable film, comprising:
a water soluble film forming agent;
a cosmetically acceptable plasticizer; and
a thickener.

2. The personal care dissolvable film of claim 1, wherein the film forming composition contains less than 5 wt. % surfactant.

3. The personal care dissolvable film of claim 1, wherein the film has a dissolution rate of less than 10 seconds.

4. The personal care dissolvable film of claim 1, wherein the thickener is at least one of carboxyvinyl polymers, polyacrylates & polymethacrylates, polyacrylamides, 2-acrylamido-2-methylpropanesulfonic acid polymers and copolymers, emulsified crosslinked anionic copolymers of acrylamide and AMPS, polysaccharide biopolymers, celluloses, starches, associative polymers, or water-soluble vinyl polymers.

5. The personal care dissolvable film of claim 1, wherein the thickener is a mixture of polyacrylamide, C₁₃₋₁₄ isoparaffin, and LAURETH-7.

6. The personal care dissolvable film of claim 1, wherein the thickener is a mixture of sodium polyacrylate/dimethicone/cyclopentasiloxane/tri-deceth-6/PEG-PPG-18/18 dimethicone.

7. The personal care dissolvable film of claim 1, wherein the thickener is an acrylates/C10-30 alkyl acrylate crosspolymer.

8. The personal care dissolvable film of claim 1, wherein the thickener is a mixture of at least two of a mixture of polyacrylamide, C₁₃₋₁₄ isoparaffin, and LAURETH-7, a mixture of sodium polyacrylate/dimethicone/cyclopentasiloxane/tri-deceth-6/PEG-PPG-18/18 dimethicone, and an acrylates/C10-30 alkyl acrylate crosspolymer.

9. The personal care dissolvable film of claim 1, wherein the thickener is present in an amount from about 0.01% to about 10% by weight of the composition.

10. The personal care dissolvable film of claim 1, wherein the thickener is present in an amount from about 0.1% to about 4% by weight of the composition.

11. The personal care dissolvable film of claim 7, wherein the thickener is present in an amount from about 0.1% to about 0.2% by weight of the composition.

12. The personal care dissolvable film of claim 6, wherein the thickener is present in an amount from about 1% to about 2% by weight of the composition.

13. The personal care dissolvable film of claim 1, wherein the plasticizer is a polyalcohol.

14. The personal care dissolvable film of claim 1, wherein the plasticizer is present in an amount from about 0.01% to about 5% by weight of the film forming composition.

15. The personal care dissolvable film of claim 1, wherein the water soluble film forming agent is a cellulose ether based polymer, polyethylene oxide, or mixtures thereof.

16. The personal care dissolvable film of claim 1, wherein the water soluble film forming agent is at least one of methylcellulose, hydroxypropyl methylcellulose, hydroxyethyl cellulose, cationic hydroxyethyl cellulose, hydrophobically modified hydroxyethyl cellulose, or cationic hydrophobically modified hydroxyethyl cellulose.

17. The personal care dissolvable film of claim 1, wherein the water soluble film forming agent is hydroxypropyl methylcellulose.

18. The personal care dissolvable film of claim 1, wherein the water soluble film forming agent is present in an amount from about 5% to about 20% by weight of the film forming composition.

19. The personal care dissolvable film of claim 1, further comprising a detackifying agent comprising at least one of dimethicone crosspolymer, calcium aluminum borosilicate spheres, or silicone resin particles including siloxane bonds and silicone groups bonded to methyl groups.

20. The personal care dissolvable film of claim 19, wherein the detackifying agent is present in an amount from about 0.1% to about 5% by weight of the film forming composition.

21. The personal care dissolvable film of claim 1, further comprising a pigment.

22.-23. (canceled)
